(19) United States
(12) Patent Application Publication
(10) Pub. No.: US 2021/0024927 A1
(43) Pub. Date: Jan. 28, 2021

(54) CHIMERIC PROMOTER WITH HIGH TRANSCRIPTIONAL ACTIVITY IN T-CELLS

(71) Applicants: Shanghai Cell Therapy Research Institute, Shanghai (CN); Shanghai Cell Therapy Group Co., Ltd., Shanghai (CN)

(72) Inventors: Qijun QIAN, Shanghai (CN); Huajun JIN, Shanghai (CN); Chao WANG, Shanghai (CN); Zhou HE, Shanghai (CN); Linfang LI, Shanghai (CN); Juanjuan SUN, Shanghai (CN); Chen HUANG, Shanghai (CN)

(73) Assignees: Shanghai Cell Therapy Research Institute, Shanghai (CN); Shanghai Cell Therapy Group Co., Ltd., Shanghai (CN)

(21) Appl. No.: 16/958,625

(22) PCT Filed: Dec. 28, 2018
(86) PCT No.: PCT/CN2018/124687

§ 371 Date: Oct. 13, 2020

(30) Foreign Application Priority Data
Dec. 29, 2017 (CN) 201711476630.6

(51) Int. Cl. C12N 15/113; C12N 15/63; C12N 5/0783; C12N 5/10

(52) U.S. Cl. CPC C12N 15/113; C12N 15/63; C12N 2830/15; C12N 5/10; C12N 5/0636

(57) ABSTRACT

Provided is a chimeric promoter with a high transcriptional activity in T-cells. The chimeric promoter consists of an enhancer sDTS and a promoter operably linked to the enhancer. The sequence of the enhancer is as shown in SEQ ID NO: 1. The enhancer is located at the 5′ or 3′ terminus of the promoter. Also provided are nucleic acid constructs, vectors and T-cells comprising the chimeric promoter. The chimeric promoter can drive the efficient expression of exogenous genes in T-cells.

### CROSS REFERENCE TO RELATED APPLICATION

This application is a national stage application under 35 U.S.C. § 371 of International Application No. PCT/CN2018/124687, filed internationally on Dec. 28, 2018, which claims priority to Chinese Patent Application No. 201711476630.6 filed Dec. 29, 2017, the content of each of which is incorporated by reference in its entirety.

### INCORPORATION BY REFERENCE OF SEQUENCE LISTING

The present application is being filed along with a Sequence Listing in electronic format. The Sequence Listing is provided as a file entitled 699532000400SeqList.txt, created Oct. 13, 2020, which is 22,651 bytes in size. The information in the electronic format of the Sequence Listing is incorporated by reference in its entirety.

### TECHNICAL FIELD

The disclosure belongs to the field of molecular biology, and relates to a chimeric promoter with high activity in T cells and uses thereof. The promoter is a synthetic chimeric promoter and has high activity in T cells. The disclosure also relates to recombinant vectors and recombinant viruses comprising the promoter and uses of the promoter in transgenic T cell therapy through controlling exogenous gene.

### BACKGROUND

Cancer has been studied for a hundred years, but so far it has not been completely conquered. With the emergence of surgical treatment, chemotherapy, radiotherapy and other treatments, the survival rate of cancer patients has been greatly improved, but these therapies are not enough to save tens of millions of cancer patients in the world. With the vigorous development of biotechnology, biotherapy, as a new treatment method, came into being, and has become the fourth largest therapy in the field of cancer treatment.

Biotherapy includes a variety of models, such as immunotherapy (cells, cytokines, antibodies, vaccines, etc.), gene therapy, regulatory angiogenesis therapy, small molecule targeted drugs and stem cell and tissue engineering regenerative medicine. Among them, cancer immunotherapy has been widely concerned by academic institutions and pharmaceutical companies since it was selected as one of the six most noteworthy scientific fields of Science in 2013.

Cancer immunotherapy mainly includes adoptive cell therapy, immunomodulators, tumor vaccines and immune checkpoint blockers. Among them, immune checkpoint blockers are effective in the treatment of many cancers, especially malignant and chemoresistant cancers. Four kinds of immune checkpoint blockers have been approved by FDA. In addition, in the field of adoptive cell therapy, especially the new generation CAR-T cell therapy technology has shown amazing therapeutic effect for blood tumors, including leukemia and lymphoma. At present, the preparation of CAR-T cells is mainly based on viral and non-viral expression systems, and viral vectors are likely to cause cell replication and insertional mutagenesis, which may lead to biosafety problems. In 2013, Cooper Laboratory of MD Anderson Cancer Center used a non-viral transfection method, namely sleeping beauty (SB) transposon system, to modify gene to obtain CD19 antigen-specific CAR-T cells.

Promoter is a component of a gene, usually located upstream of the 5′end of a structural gene. It is a DNA sequence recognized and bound by RNA polymerase and from which the transcription starts. Promoter is one of the important factors that affect the transgenic expression efficiency, and choosing a highly efficient promoter is the key to efficiently expression of an exogenous gene.

According to their transcriptional patterns, promoters can be divided into three types: constitutive promoters, tissue or organ specific promoters and inducible promoters.

Constitutive promoters refer to promoters that under their regulation there is no significant difference in gene expression in different tissues, organs and development stages, and are therefore called constitutive promoters. Constitutive promoters commonly used in mammals include those from viruses: murine or human cytomegalovirus (CMV) promoters (mCMV and hCMV, respectively), monkey vacuolar virus SV40 promoter; those from natural human genome: EF1α promoter, ubiquitin promoter (Ubi for short), β-actin promoter, PGK-1 promoter, Rosa26 promoter, HSP70 promoter, GAPDH promoter, eIF4A1 promoter, Egr1 promoter, FerH promoter, SM22α promoter, Endothelin-1 promoter, etc.

In tumor immunotherapy, it is very important to maintain the highly efficient and stable expression of exogenous genes. However, although the transient expression activity of some constitutive promoters from viruses is high (such as CMV promoter), such expression is easy to be stop due to epigenetic modification; while some natural constitutive promoters from human or tumor specific promoters are stable in expression, but their expression activity is relatively low, which is difficult to meet the needs of immunotherapy. Therefore, the researchers designed and constructed a series of artificial chimeric promoters, which comprised some cis-acting elements, mainly including promoter core sequences which could stabilize expression, and upstream enhancers or downstream introns that can enhance the expression efficiency. A Represented artificial chimeric promoters is the chimeric promoter CAG (including human CMV enhancer-chicken β-actin promoter-rabbit β-globin intron), which has been widely used in the expression of exogenous genes.

Enhancer refers to the DNA sequence that increases the transcription frequency of genes linked with it Enhancers increase the transcription of downstream genes through promoters. Effective enhancers can be located at the 5′end, at the 3′ end or in an intron of a gene. Enhancers have significant effect, which generally can increase the gene transcription frequency by 10˜200-folds, some even up to thousands-folds.

### SUMMARY

To solve the technical problem that the transcription intensity of the promoter in the expression vector is not high enough in the prior art, the inventor designed and constructed a series of novel chimeric promoters through a lot of experiments and creative labor, and screened out a group of promoters that can express efficiently in T cells. The promoter is especially suitable for driving the high expression of exogenous genes (such as full-length antibody genes) in T cells.

Therefore, one aspect of the disclosure provides an enhanced promoter sequence (i.e. chimeric promoter), the enhanced promoter sequence consisting of an enhancer sDTS and a promoter operably linked to the enhancer, wherein the sequence of the enhancer sDTS is as shown in SEQ ID NO: 1.

In one or more embodiments, the promoter is selected from the group consisting of: an EF1α promoter, a PGK promoter, a β-actin promoter, an hCMV promoter, an EEF2 promoter, a CAG promoter, a U6 promoter and a SV40 promoter.

In one or more embodiments, the promoter is an EF1α promoter, an hCMV promoter or a β-actin promoter, preferably an EF1α promoter.

In one or more embodiments, the enhancer is located at the 5′end or 3′ end of the promoter, preferably located at the 5′end of the promoter.

In one or more embodiments, the nucleic acid sequence of the enhanced promoter sequence consists of the nucleic acid sequence as shown in SEQ ID NO: 1 and the nucleic acid sequence as shown in SEQ ID NO: 3.

The disclosure also provides a nucleotide construct comprising the enhanced promoter sequence described herein.

In one or more embodiments, the nucleic acid construct is an expression cassette, which sequentially comprises, from the 5′ end to the 3′ end, the enhanced promoter sequence, the coding sequence of a protein of interest and a transcription terminator, that are operably linked.

The disclosure also provides a vector comprising the enhanced promoter sequence described herein.

In one or more embodiments, the vector is a eukaryotic expression vector, selected from the group consisting of: a transient expression vector, a virus expression vector and a transposition vector.

In one or more embodiments, the vector is a vector for integrating the expression cassette of a gene of interest into the genome of a host cell, preferably is a transposition vector.

In one or more embodiments, the transposition vector comprises a transposable element selected from the group consisting of: piggyback, sleeping beauty, frog prince, Tn5 and Ty.

In one or more embodiments, the transposition vector sequentially comprises the enhanced promoter sequence, one or more restriction sites or optional coding sequence(s) of interest and a transcription termination sequence, that are operably linked between the 5′ LTR and the 3′ LTR.

In one or more embodiments, a coding sequence of a transposase and its promoter sequence are operably linked at the 3′ end of the 3′ LTR.

In one or more embodiments, the vector also comprises one or more selectable markers.

In one or more embodiments, the coding sequence of interest is a coding sequence of an antibody.

In one or more embodiments, the antibody is selected from the group consisting of: a PD-1 antibody, a PD-L1 antibody, a CTLA4 antibody, a CD40 antibody, a CD40L antibody, a CD47 antibody, a CD137 antibody, a CD28 antibody, a CD27 antibody, a OX40 antibody, a DNAM-1 antibody, a GITR antibody, a ICSOS antibody, a 2B4 antibody, a CD160 antibody, a TIM3 antibody, a LAGS antibody, a BTLA antibody and a TIGIT antibody; preferably a PD-1 single-chain antibody.

In one or more embodiments, the restriction site is selected from the group consisting of: AscI restriction site, XbaI restriction site, PvuI restriction site, EcoRI restriction site and SalI restriction site.

In one or more embodiments, the nucleic acid sequence of the vector is as shown in SEQ ID NO: 12.

The disclosure also provides the use of the enhanced promoter sequence described herein in driving the expression of an exogenous gene (such as a full-length antibody gene) in a T cell.

### BRIEF DESCRIPTION OF DRAWINGS

FIG. 1: A schematic diagram of the structures of the expression vectors carrying the nucleotide sequence of each promoter.

FIG. 2A-2B: The fluorescence intensities (2A) and positive rates (2B) of the expressions of pNB328-E-EGFP, pNB338A-E-EGFP and pNB338B-E-EGFP plasmids in T cell lines.

FIG. 3: The Nluc fluorescence values of the expressions of pNB328-E-Nluc, pNB338B-E-Nluc, pNB328-hC-Nluc, pNB338B-hC-Nluc, pNB328-β-Nluc and pNB338B-β-Nluc plasmids in T cell lines.

FIG. 4: A comparison of the expressions of PD-1 antibodies expressed by pNB328-E-m279V, pNB338B-E-m279V, pNB328-hC-m279V, pNB338B-hC-m279V, pNB328-β-m279V and pNB338B-β-m279V plasmids.

FIG. 5: The expressions of EGFP protein in CHO cells mediated by EF1α and sDTS+EF1α promoters.

### DETAILED DESCRIPTION

Some terms involved in the disclosure are explained below. It should be understood that, unless otherwise defined herein, terms used herein have meanings generally recognized in the art.

In the present disclosure, the term “expression cassette” refers to the complete elements required to express a gene, including operably linked promoter(s) and gene coding sequence(s).

The term “coding sequence” refers to a part of a nucleic acid sequence that directly determines the amino acid sequence of its protein product. The boundaries of a coding sequence are usually determined by a ribosome binding site closely adjacent to the upstream of the open reading frame at mRNA 5′ end (for prokaryotic cells) and a transcription termination sequence closely adjacent to the downstream of the open reading frame at mRNA 3′ end. The coding sequence can include, but is not limited to, DNA, cDNA and recombinant nucleic acid sequences.

The term “single-chain antibody” (scFv) refers to an antibody fragment formed by linking the amino acid sequences of antibody light chain variable region (V_{L} region) and the amino acid sequences of heavy chain variable region (V_{H} region) with hinge, which has antigen-binding ability. In some embodiments, the single-chain antibody (scFv) of interest is from an antibody of interest. The antibodies of interest may be human antibodies, including human-murine chimeric antibodies and humanized antibodies. The antibody may be a secretory antibody or a membrane-anchored antibody.

The term “operably linked” or “in operably linkage” refers to DNA regulatory sequences (such as enhancers, promoters, etc) linked to the coding sequence of a protein of interest in a manner that allows the expression of the coding sequence of the protein of interest.

The disclosure improves the expression intensity of the gene driven by a promoter by modifying the promoter activity, so as to obtain a high-efficiency expression of an exogenous gene, especially the full-length antibody genes in T cells.

The enhanced promoter sequence of the disclosure consists of an enhancer sDTS and a promoter operably linked to the enhancer, wherein the sequence of the enhancer sDTS is as shown in SEQ ID NO: 1.

The promoter in the enhanced promoter sequence of the disclosure can be any promoter sequence conventionally used in the art to express a exogenous gene in T cells, including but not limited to a EF1α promoter, a PGK promoter, a β-actin promoter, a hCMV promoter, a EEF2 promoter, a CAG promoter, a U6 promoter and a SV40 promoter; preferably, the promoter is a EF1α promoter, a hCMV promoter and a β-actin promoter, more preferably a EF1α promoter. The nucleotide sequence of an exemplary EF1α promoter can be as shown in SEQ ID NO: 3; the nucleotide sequence of an exemplary hCMV promoter can be as shown in SEQ ID NO: 4; the nucleotide sequence of an exemplary β-actin promoter can be as shown in SEQ ID NO: 5.

In some embodiments, the enhanced promoter sequence of the disclosure consists of SEQ ID NO: 1 and SEQ ID NO: 3, 4 or 5, preferably consists of SEQ ID NO: 1 and SEQ ID NO: 3.

The disclosure includes nucleic acid construct comprising the enhanced promoter sequence described herein.

In some embodiments, the nucleic acid is an expression cassette comprising the enhanced promoter sequence described herein and a coding sequence of a protein of interest. The expression cassette usually comprises a transcription termination sequence (i.e. transcription terminator), which is a sequence recognized by host cells to terminate transcription. The transcription termination sequence is operably linked to the 3′ end of the coding sequence described herein. Any terminator that functions in the selected host cell can be used in the present disclosure, including but not limited to the SV40 polyA transcription termination sequence.

In some embodiments, the nucleic acid construct is a vector. The vector usually includes but is not limited to a plasmid, a phage particle, a phage derivative, an animal virus and a cosmid. The vector may be an expression vector, including a transient expression vector, a viral expression vector and a transposition vector. The vector is preferably a eukaryotic expression vector. Viruses that can be used as vectors include but are not limited to retroviruses, adenoviruses, adeno-associated viruses, herpesviruses and lentiviruses.

Generally, a suitable vector comprises at least one origin of replication that function in the host cell, convenient restriction site(s), and one or more selectable markers.

The restriction site includes but is not limited to an AscI restriction site, an XbaI restriction site, a PvuI restriction site, an EcoRI restriction site and a SalI restriction site. Generally, some of the restriction sites in the vector is located between the enhanced promoter sequence of the present disclosure and the transcription termination sequence, which is used to cut the vector there and insert a coding sequence of a protein of interest, so that the coding sequence can be operably linked to the enhanced promoter sequence of the disclosure and the transcription termination sequence.

The selectable markers include any one or both of selectable marker genes and reporter genes, so as to identify and select the expressing cells from the cell population infected by virus vector(s). Useful selectable marker genes include, for example, antibiotic resistance genes, such as neo and the like. Suitable reporter genes can include a gene encoding luciferase, β-galactosidase, chloramphenicol acetyltransferase, secretory alkaline phosphatase or green fluorescent protein.

In some embodiments, the vector is a vector for integrating an expression cassette of a gene of interest into the genome of a host cell, preferably is a transposition vector. In some embodiments, the transposition vector is a eukaryotic expression vector comprising a transposable element selected from the group consisting of: piggyback, sleeping beauty, frog prince, Tn5 and Ty. Such transposition vector comprises the 5′ inverted terminal repeat (5′ LTR) of the corresponding transposon and the 3′ inverted terminal repeat (3′ LTR) of the corresponding transposon. The transposase can be a transposase from piggybac, sleeping beauty, frog prince, Tn5 or Ty transposon system. When transposases from different transposon systems are used, the sequences of the 5′ LTR and 3′ LTR in the vector are changed accordingly into the sequences suitable for the transposon system, which can be easily determined by those skilled in the art.

In some embodiments, the transposase is a transposase from piggyback transposon system. Therefore, in these embodiments, the transposon 5′ inverted terminal repeat and 3′ inverted terminal repeat are the 5′ inverted terminal repeat and 3′ inverted terminal repeat of piggybac transposon, respectively. In some embodiments, the transposon 5′ inverted terminal repeat is as shown in SEQ ID NO: 1 of CN201510638974.7 (the contents of which are incorporated herein by reference). In some embodiments, the transposon 3′ inverted terminal repeat is as shown in SEQ ID NO: 4 of CN201510638974.7. In some embodiments, the piggyback transposase is a transposase comprising c-myc nuclear positioning signal coding sequence. In some embodiments, the coding sequence of the piggybac transposase is as shown in SEQ ID NO: 5 of CN201510638974.7.

Promoters of the transposase coding sequences may be various promoters known in the art for controlling the expression of the transposase coding sequences. In some embodiments, CMV promoter is used to control the expression of the transposase coding sequence. The sequence of CMV promoter can be as shown in SEQ ID NO: 6 of CN201510638974.7.

In some embodiments, the pNB328 vector disclosed in CN201510638974.7 is used as the skeleton of the vector of the disclosure, but the EF1α promoter contained in the vector is replaced by the enhanced promoter sequence described herein.

In some embodiments, the vector of the disclosure is an empty vector, that is, it does not comprise the coding sequence of a protein of interest. Generally, such empty vectors sequentially comprise the enhanced promoter sequence described herein, one or more restriction endonuclease sites and a transcription termination sequence, which are used to link the coding sequence of a protein of interest between the enhanced promoter sequence and the transcription termination sequence by enzymatic digestion. In some embodiments, the vector of the disclosure is a vector in which the coding sequence of a protein of interest is inserted between the enhanced promoter sequence described herein and a transcription termination sequence, preferably is a transposition vector. Such a vector sequentially comprises the enhanced promoter sequence described herein, the coding sequence of the protein of interest and the transcription termination sequence between the 5′ LTR and 3′ LTR, preferably also comprises a coding sequence of a transposase and its promoter sequence at the 3′ end of the 3′ LTR.

The disclosure also includes complementary sequences of each nucleotide sequence described herein. The polynucleotide sequences herein can be in the form of DNA or RNA.

The nucleotide sequences described here can generally be obtained by PCR amplification. Specifically, sequences of interest can be amplified by use of primers designed based on the nucleotide sequences disclosed herein, and commercially available cDNA libraries or cDNA libraries prepared according to conventional methods known to those skilled in the art as templates. When the sequence is long, it is often necessary to carry out two or more PCR amplifications, and then the amplified fragments are spliced together in the correct order. In some embodiments, some nucleotide sequences of the disclosure can be synthesized by artificial synthesis method under suitable conditions.

In this disclosure, the proteins of interest can be various proteins well known in the art, including but not limited to enzymes, antibodies, and other proteins with desired functions. Preferably, proteins of interest are proteins well-known in the art that need to be expressed in T cells, such as various antibodies with anti-tumor effect, including single-chain antibody, or chimeric antigen receptor (CAR).

The enhanced promoter sequence of the disclosure is particularly suitable for driving the expression of genes of various antibodies, preferably full-length antibodies, in T cells. Exemplary antibodies include but are not limited to a PD-1 antibody, a PD-L1 antibody, a CTLA4 antibody, a CD40 antibody, a CD40L antibody, a CD47 antibody, a CD137 antibody, a CD28 antibody, a CD27 antibody, a OX40 antibody, a DNAM-1 antibody, a GITR antibody, a ICSOS antibody, a 2B4 antibody, a CD160 antibody, a TIM3 antibody, a LAGS antibody, a BTLA antibody and a TIGIT antibody.

In some embodiments, the expression vector of the disclosure comprises the enhanced promoter sequence described herein and a coding sequence of PD-1 antibody, which is used to express PD-1 antibody in T cells or to integrate the expression cassette of PD-1 antibody into the genome of T cells.

The vector of the disclosure can be transferred into cells of interest by conventional transfection methods, including but not limited to virus transduction, microinjection, particle bombardment, gene gun transformation and electroporation. In some embodiments, the vector described herein is transfected into the cells of interest by electroporation.

The cells of interest can be various T cells well known in the art, including but not limited to peripheral blood T lymphocytes, cytotoxic T cells (CTLs), helper T cells, inhibitory/regulatory T cells, γδT cells and cytokine-induced killer cells (CIK), tumor infiltrating lymphocytes (TIL), or T cells of mixed cell populations. In some embodiments, the T cells can be derived from PBMC of patients with B cell malignancies. In some embodiments, the T cells are primarily cultured T cells.

Therefore, in some embodiments, the disclosure provides the use of the enhanced promoter sequence described herein in driving the expression of exogenous gene(s) (such as full-length antibody genes) in T cells.

In some embodiments, the disclosure also provides a T cell comprising the enhanced promoter sequence or nucleic acid construct or vector described herein. Preferably, the genome of the T cell is integrated with an expression cassette, and the enhanced promoter sequence described herein is used in the expression cassette as a promoter to drive the expression of an exogenous gene of interest. More preferably, the genome of the T cell of the disclosure is integrated with an expression cassette comprising the enhanced promoter sequence described herein and the coding sequence of PD-1 antibody which is operably linked to the promoter sequence. In some embodiments, compared with the control T cells without the enhancer sequence described herein but containing the same promoter sequence, the expression amount of the protein of interest of the T cells according to the disclosure is at least 1.1 times, at least 1.2 times or at least 1.5 times of the expression amount of the protein of interest of the control T cells, as measured under the same conditions.

In some embodiments, the amino acid sequence of the PD-1 antibody described herein is as shown in SEQ ID NO: 11, and its exemplary coding sequence is as shown in SEQ ID NO: 10.

The embodiments of the present disclosure illustrated by way of specific examples below. Those skilled in the art will understand that these examples are merely exemplary and should not be considered as limiting the scope of the present disclosure. The experimental methods without specifying the specific technology or conditions in the following examples generally used the conventional technology or conditions, such as those described in J. Sambrook et al., Molecular Cloning: A Laboratory Manual (3rd ed.), translated by Huang Peitang et al., Science Press, or followed the manufacturer's recommendation. The used reagents or instruments without specifying the manufacturer are all conventional products that are commercially available.

### Example 1: Construction of Expression Vector Carrying Nucleotide Sequence of Each Promoter

1. Construction of Enhanced Green Fluorescent Protein EGFP Expression Vector

Sequence sDTS-LTR (SEQ ID NO: 2) was synthesized by Shanghai Generay Biotech Co., Ltd with AscI restriction site introduced into its upstream and XbaI restriction site inserted into its downstream. The sequence was inserted into pNB328 vector (pNB328 vector comprises EF1α promoter; its structure and sequence are as described in CN201510638974.7, the whole content of which is incorporated herein by reference) which was double digested by AscI and XbaI, to construct the recombinant plasmid, named pNB338A-E.

Sequence sDTS (SEQ ID NO: 1) was synthesized by Shanghai Generay Biotech Co., Ltd with XbaI restriction site introduced into its upstream and PvuI restriction site inserted into its downstream. The sequence was inserted into pNB328 vector (which was double digested by XbaI and PvuI, to construct the recombinant plasmid, named pNB338B-E.

Green fluorescent protein EGFP gene sequence (its nucleic acid sequence is as shown in SEQ ID NO: 8, its amino acid sequence is as shown in SEQ ID NO: 9) was synthesized by Shanghai Generay Biotech Co., Ltd with EcoRI restriction site introduced into its upstream and SalI restriction site inserted into its downstream. The sequence was respectively inserted into pNB328 vector, pNB338A-E vector and pNB338B-E vector which were double digested by EcoRI and SalI, to construct the recombinant plasmids, named pNB328-E-EGFP, pNB338A-E-EGFP and pNB338B-E-EGFP, respectively.

2. Construction of Luciferase Reporter Gene Expression Vector Carrying Each Promoter

The coding sequence of nano luciferase (NLuc for short) gene (its nucleic acid sequence is as shown in SEQ ID NO: 6, its amino acid sequence is as shown in SEQ ID NO: 7) in pNL1.3.CMW[secNluc-CMV] plasmid was synthesized by Shanghai Generay Biotech Co., Ltd with EcoRI restriction site introduced into its upstream and SalI restriction site inserted into its downstream. The sequence was respectively inserted into pNB328-E vector and pNB338B-E vector which were double digested by EcoRI and SalI, to construct the recombinant plasmids, named pNB328-E-NLuc and pNB338B-E-NLuc, respectively.

The nucleotide sequences of hCMV promoter and β-Actin promoter as shown in SEQ ID NO: 4 and SEQ ID NO: 5 were synthesized by Shanghai Generay Biotech Co., Ltd with XbaI introduced into its upstream and EcoRI restriction site introduced into its downstream. The sequences were inserted into pNB328-E-NLuc vectors which were double digested by XbaI and EcoRI (replacing the EF1α promoter in the original pNB328-E-NLuc vector), to construct the recombinant plasmids, named pNB328-hC-NLuc and pNB328-β-NLuc, respectively. The sequences were also inserted into pNB338B-E-NLuc vectors which were double digested by XbaI and EcoRI, to construct the recombinant plasmids, named pNB338B-hC-NLuc and pNB338B-β-NLuc, respectively.

3. Construction of PD-1 Antibody Expression Vector Carrying Each Promoter

PD-1 antibody nucleotide sequence (its nucleic acid sequence is as shown in SEQ ID NO: 10, its amino acid sequence is as shown in SEQ ID NO: 11) was synthesized by Shanghai Generay Biotech Co., Ltd with EcoRI restriction site introduced into its upstream and SalI restriction site inserted into its downstream. The sequence was inserted into pNB328-E-NLuc, pNB328-hC-NLuc, pNB328-β-NLuc, pNB338B-E-NLuc, pNB338B-hC-NLuc and pNB338B-β-NLuc vectors which were double digested by EcoRI and SalI respectively, to construct the recombinant plasmids, named pNB328-E-m279V, pNB328-hC-m279V, pNB328-β-m279V, pNB338B-E-m279V, pNB338B-hC-m279V and pNB338-β-m279V, respectively.

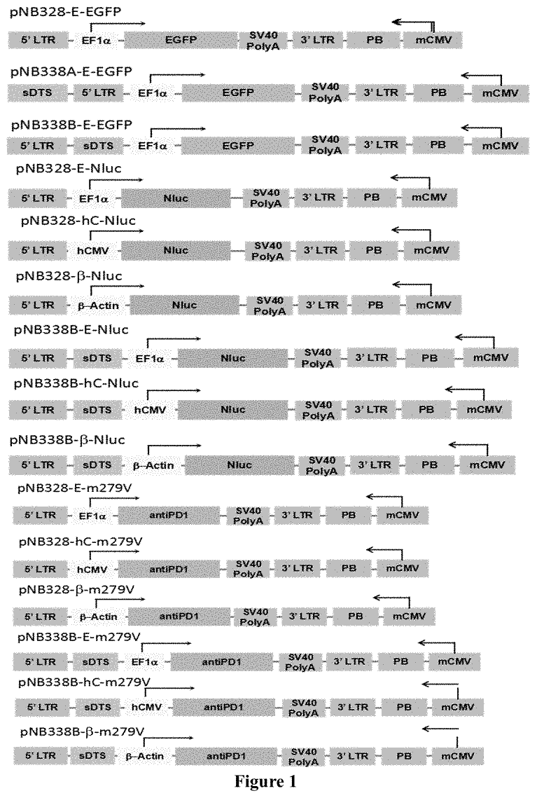

The schematic diagram of the structure of each expression vector is shown in FIG. 1.

### Example 2: Construction of T Cells

Peripheral blood mononuclear cells (PBMCs) were isolated from donor blood by Filcoll isolation method. PBMCs were cultured adherently for 2-4 h, wherein the non-adherent suspension cells were the initial T cells. The suspension cells were collected into a 15 ml centrifuge tube and centrifuged at 1200 rmp for 3 min, the supernatant was discarded, normal saline was added, the mixture was centrifuged at 1200 rmp for 3 min, normal saline was discarded and the procedure was repeated.

15 of 1.5 ml centrifugation tubes with 5×10⁶ cells added into each tube were numbered 1-15 and centrifuged at 1200 rmp for 3 min, the supernatant was discarded, 100 ul of the electroporation reagent from the Electroporation Kit (Lonza company) was added into each tube in proportion, and 6 ug of the above plasmids was added into 1-15 tubes respectively, and the cells were re-suspended; the mixed liquid was transferred to electroporation cuvettes and placed into the electroporator, the required program was selected and the electrical pulse was produced; the electroporated cell suspension was transferred to six-well plates supplemented with medium (AIM-V medium containing 2% FBS) using the micropipette in the kit, evenly mixed and cultured at 37° C., 5% CO₂ in an incubator, after 6 hours, stimulating factor IL-2 and CD3 antibody, CD28 antibody were added, after 3-4 days of culture at 37° C., 5% CO₂, the growth of T cells was observed, and T cells expressing EGFP, Nluc and PD-1 antibody were obtained.

### Example 3: Comparison and Identification of EGFP Expressed by pNB328-E-EGFP, pNB338A-E-EGFP and pNB338B-E-EGFP Plasmids

The activated T cells modified with pNB328-E-EGFP, pNB338A-E-EGFP and pNB338B-E-EGFP plasmids obtained from Example 2 were observed under fluorescence microscope on the 8^{th} and 18^{th} days after electroporation, and 5×10⁵ cell precipitates were collected respectively and washed by PBS for 2 times, 400 ul PBS was added before transferring the cells into flow cytometry tubes, and the flow cytometry tubes were loaded onto the flow cytometry device for detection.

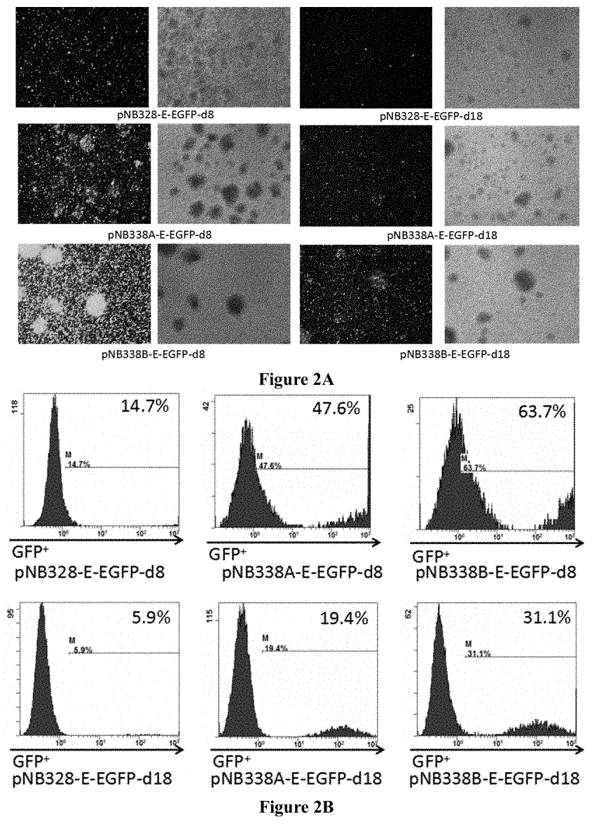

Results are shown in FIGS. 2A and 2B and the following Table 1, both the positive rate and fluorescence intensity of the expression of pNB338B-E-EGFP plasmid were better than those of pNB338A-E-EGFP plasmid, while the positive rate and fluorescence intensity of the expression of pNB338A-E-EGFP plasmid were better than those of pNB328-E-EGFP plasmid. This indicates that the expression of the plasmid containing sDTS sequence is stronger than that of the plasmid without sDTS sequence, and the expression of the plasmid in which sDTS sequence is located before EF1α promoter and after 5′ LTR (pNB338B-E-EGFP) is stronger.

TABLE 1

| Cell type | Culture days | Fluorescence intensity |
|---|---|---|
| pNB328-E-EGFP T cell | 8 | 188 |
| pNB338A-E-EGFP T cell | 8 | 360 |
| pNB338B-E-EGFP T cell | 8 | 510 |
| pNB328-E-EGFP T cell | 18 | 76 |
| pNB338A-E-EGFP T cell | 18 | 179 |
| pNB338B-E-EGFP T cell | 18 | 318 |

### Example 4: Comparison of Nluc Expressed by pNB328-E-Nluc, pNB338B-E-Nluc, pNB328-hC-Nluc, pNB338B-hC-Nluc, pNB328-β-Nluc and pNB338B-β-Nluc Plasmids

2×10⁶ of each of the activated T cells modified with pNB328-E-Nluc, pNB338B-E-Nluc, pNB328-hC-Nluc, pNB338B-hC-Nluc, pNB328-β-Nluc and pNB338B-β-Nluc plasmids obtained from Example 2 were collected on the 7^{th} and 17^{th} days, placed into 6-well plates supplemented with 3 ml AIM-V medium at the density of 2×10⁶ cells/well, and incubated at 37° C., 5% CO₂ in an incubator. After 24 h of culture, the cell supernatant was collected and stored at −20° C. for later use. The fluorescence value of the novel fluorescein Nluc secreted into the cell supernatant was measured on a Microplate Reader.

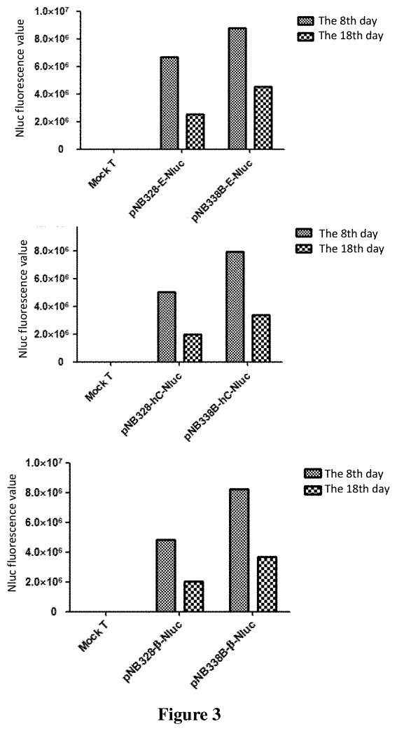

Results are shown in FIG. 3 and the following Tables 2-4, for different promoters (EF1α/hCMV/β-Actin), all the Nluc fluorescence values expressed by the plasmids (pNB338B-E-Nluc, pNB338B-hC-Nluc, pNB338B-β-Nluc) containing sDTS sequence were higher than those of the plasmids (pNB328-E-Nluc, pNB328-hC-Nluc, pNB328-β-Nluc) with the same promoter but without sDTS sequence.

TABLE 2

| Cell type | Culture days | Nluc fluorescence value |
|---|---|---|
| Mock T cell | 8 | 286 |
| Mock T cell | 18 | 300 |
| pNB328-E-Nluc T cell | 8 | 6666082 |
| pNB328-E-Nluc T cell | 18 | 2528848 |
| pNB338B-E-Nluc T cell | 8 | 8772142 |
| pNB338B-E-Nluc T cell | 18 | 4511353 |

TABLE 3

| Cell type | Culture days | Nluc fluorescence value |
|---|---|---|
| Mock T cell | 8 | 300 |
| Mock T cell | 18 | 332 |
| pNB328-hC-Nluc T cell | 8 | 5056150 |
| pNB328-hC-Nluc T cell | 18 | 1984001 |
| pNB338B-hC-Nluc T cell | 8 | 7935584 |
| pNB338B-hC-Nluc T cell | 18 | 3382779 |

TABLE 4

| Cell type | Culture days | Nluc fluorescence value |
|---|---|---|
| Mock T cell | 8 | 293 |
| Mock T cell | 18 | 315 |
| pNB328-β-Nluc T cell | 8 | 4841137 |
| pNB328-β-Nluc T cell | 18 | 2028848 |
| pNB338B-β-Nluc T cell | 8 | 8235377 |
| pNB338B-β-Nluc T cell | 18 | 3680633 |

The results also indicate that, regardless of the expressed genes, all the expression plasmids containing sDTS sequence mediated by different promoters are better than the plasmids without sDTS sequence.

### Example 5: Comparison of the Expression of PD-1 Antibody Expressed by pNB328-E-m279V, pNB338B-E-m279V, pNB328-hC-m279V, pNB338B-hC-m279V, pNB 32843-m279V and pNB338B-β-m279V Plasmids

2×10⁶ of each of the activated T cells modified with pNB328-E-m279V, pNB338B-E-m279V, pNB328-hC-m279V, pNB338B-hC-m279V, pNB328-β-m279V and pNB338B-β-m279V plasmids obtained from Example 2 were collected on the 8^{th} and 18^{th} days, placed into 6-well plates supplemented with 3 ml AIM-V medium at the density of 2×10⁶ cells/well, and incubated at 37° C., 5% CO₂ in an incubator. After 24 h of culture, the cell supernatant was collected and stored at −20° C. for later use. A double antibody sandwich ELISA method (using human PD-1 recombinant protein to coat ELISA plates, HRP-labeled mouse anti-human IgG4 mAb to detect, and commercialized anti-PD-1 antibody as standard) was used, and the sample to be tested was diluted by 50-folds before quantitative detection.

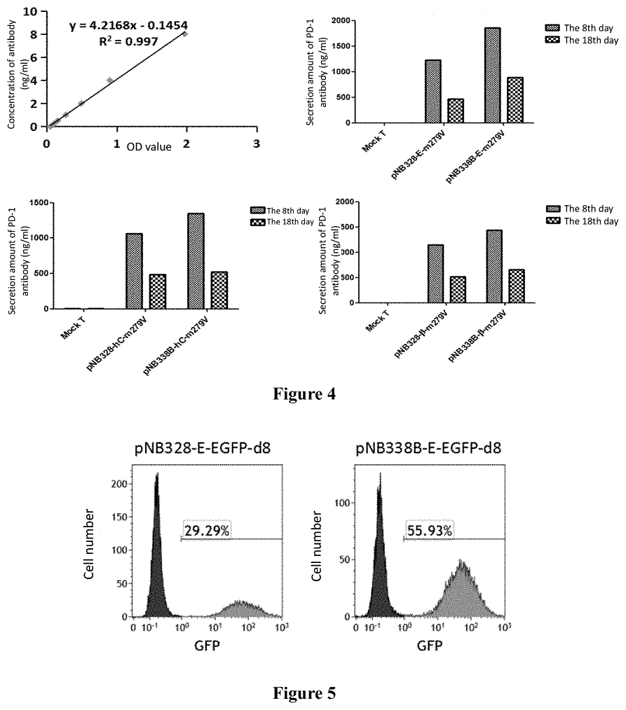

Results are shown in FIG. 4 and the following Tables 5-7, for different promoters (EF1α/hCMV/β-Actin), all the amounts of the PD-1 antibody expressed by the plasmids (pNB338B-E-m279V, pNB338B-hC-m279V, pNB338B-β-m279V) containing sDTS sequence were higher than those of the plasmids (pNB338B-E-m279V, pNB338B-hC-m279V, pNB338B-β-m279V) with the same promoter but without sDTS sequence.

TABLE 5

| Cell type | Culture days | PD1 antibody (ng/ml) |
|---|---|---|
| Mock T cell | 8 | 0.23 |
| Mock T cell | 18 | 0.13 |
| pNB328-E-m279v T cell | 8 | 1230 |
| pNB328-E-m279v T cell | 18 | 470 |
| pNB338B-E-m279v T cell | 8 | 1860 |
| pNB338B-E-m279v T cell | 18 | 890 |

TABLE 6

| Cell type | Culture days | PD1 antibody (ng/ml) |
|---|---|---|
| Mock T cell | 8 | 1.51 |
| Mock T cell | 18 | 1.73 |
| pNB328-hC-m279v T cell | 8 | 1060 |
| pNB328-hC-m279v T cell | 18 | 488 |
| pNB338B-hC-m279v T cell | 8 | 1350 |
| pNB338B-hC-m279v T cell | 18 | 522 |

TABLE 7

| Cell type | Culture days | PD1 antibody (ng/ml) |
|---|---|---|
| Mock T cell | 8 | 0.44 |
| Mock T cell | 18 | 0.49 |
| pNB328-β-m279v T cell | 8 | 1145 |
| pNB328-β-m279v T cell | 18 | 513 |
| pNB338B-β-m279v T cell | 8 | 1437 |
| pNB338B-β-m279v T cell | 18 | 660 |

The results also indicate that all the expression plasmids containing sDTS sequence mediated by different promoters are better than the plasmids without sDTS sequence. Meanwhile, the plasmids containing sDTS sequence not only have better ability to express reporter genes, but also have stronger ability to express functional proteins.

### Comparative Example 1: Expression of EGFP Protein in CHO Cells Mediated by EF1α and sDTS+EF1α Promoters

The pNB328-E-EGFP and pNB338B-E-EGFP obtained from Example 1 were used to transfect CHO cells respectively. Specifically, 2 centrifugation tubes were added with 5×10⁶ CHO cells each and centrifuged at 1200 rmp for 3 min, the supernatant was discarded, 100 ul of the electroporation reagent from electroporation kit (Lonza company) was added into each tube in proportion, and 6 ug of pNB328-E-EGFP and 6 ug of pNB338B-E-EGFP were added into each tube respectively, and the cells were re-suspended respectively; the mixed liquid was transferred to electroporation cuvettes and placed into the electroporator, the required program was selected and the electric pulse was produced. After electroporation, CHO cells were cultured in 1640/DMEM (1:1 mixed) medium containing 10% FBS at 37° C., 5% CO₂ in an incubator. On the 8^{th} day after electroporation, precipitates of 5×10⁵ cell were collected and washed by PBS for 2 times, 400 ul PBS was added before transferring the cells into flow cytometry tubes, and the flow cytometry tubes were loaded onto the flow cytometry device for detection. Results are shown in FIG. 5

On the 8^{th} day, the positive rate of the CHO cells transfected with pNB338B-E-EGFP was about 1.9 times of the positive rate of the CHO cells transfected with pNB328-E-EGFP. In Example 3, on the 8^{th} day, the positive rate of the T cells transfected with pNB338B-E-EGFP was about 4.3 times of the positive rate of the T cells transfected with pNB328-E-EGFP. This result indicates that the enhanced promoter of the disclosure can enhance the promoter transcription intensity specifically in T cells.

### Comparative Example 2: Comparison of the Expression of PD-1 Antibody Expressed by pNB338h-E-m279V, pNB338h-hC-m279V, pNB338h-β-m279V, pNB338e-E-m279V, pNB338e-hC-m279V and pNB338e-β-m279V Plasmids

The pNB338h-β-m279V, pNB338h-hC-m279V, pNB338h-β-m279V, pNB338e-β-m279V, pNB338e-hC-m279V and pNB338e-β-m279V recombinant plasmids were constructed according to the method of Example 1. Different from pNB338B-E-m279V, pNB338B-hC-m279V and pNB338B-β-m279V, hCMV enhancer was used in pNB338h-β-m279V, pNB338h-hC-m279V and pNB338h-β-m279V to replace sDTS sequence, and CD3e enhancer was used in pNB338e-β-m279V, pNB338e-hC-m279V and pNB338e-β-m279V to replace sDTS sequence.

2×10⁶ of each of the activated T cells modified with pNB338h-β-m279V, pNB338h-hC-m279V, pNB338h-β-m279V, pNB338e-β-m279V, pNB338e-hC-m279V and pNB338e-β-m279V plasmids were collected on the 8^{th} and 18th days, placed into 6-well plates supplemented with 3 ml AIM-V medium at the density of 2×10⁶ cells/well, and incubated at 37° C., 5% CO₂ in an incubator. After 24 h of culture, the cell supernatant was collected and stored at −20° C. for later use. A double antibody sandwich ELISA method (using human PD-1 recombinant protein to coat ELISA plates, HRP-labeled mouse anti-human IgG4 mAb to detect, and commercialized anti-PD-1 antibody as standard) was used, and the sample to be tested was diluted by 50-fold before quantitative detection.

Results show that, for different promoters (EF1α/hCMV/β-Actin), all the amounts of the PD-1 antibody expressed by the plasmids (pNB338B-E-m279V, pNB338B-hC-m279V, pNB338B-β-m279V) containing sDTS sequence were higher than those of the plasmids with the same promoter but containing hCMV enhancer sequence or CD3e enhancer sequence.

These results indicate that all the expression plasmids containing sDTS sequence mediated by different promoters are better than the plasmids containing other enhancer sequences. Meanwhile, the ability of the plasmid containing sDTS sequence to express a specific gene is stronger than the ability of other enhancer sequence to mediate gene expression.

1-10. (canceled)

11. An enhanced promoter sequence, wherein the enhanced promoter sequence consists of an enhancer sDTS and a promoter operably linked to the enhancer, wherein the sequence of the enhancer sDTS is as shown in SEQ ID NO: 1; and the enhancer is located at a 5′ end or a 3′ end of the promoter.

12. The enhanced promoter sequence according to claim 11, wherein the promoter is selected from the group consisting of: an EF1α promoter, a PGK promoter, a β-actin promoter, a hCMV promoter, an EEF2 promoter, a CAG promoter, a U6 promoter and a SV40 promoter.

13. The enhanced promoter sequence according to claim 12, wherein the promoter is an EF1α promoter, the nucleotide sequence of the EF1α promoter is as shown in SEQ ID NO: 3; the nucleotide sequence of the hCMV promoter is as shown in SEQ ID NO: 4; the nucleotide sequence of the β-actin promoter is as shown in SEQ ID NO: 5.

14. A nucleic acid construct, wherein the nucleic acid construct comprises the enhanced promoter sequence according to claim 11.

15. The nucleic acid construct according to claim 14, wherein the promoter is an EF1α promoter, the nucleotide sequence of which is as shown in SEQ ID NO: 3; or the promoter is a hCMV promoter, the nucleotide sequence of which is as shown in SEQ ID NO: 4; or the promoter is a β-actin promoter, the nucleotide sequence of which is as shown in SEQ ID NO: 5.

16. The nucleic acid construct according to claim 14, wherein the nucleic acid construct is an expression cassette, which sequentially contains, from the 5′ end to the 3′ end, the enhanced promoter sequence, a coding sequence of a protein of interest and a transcription terminator, that are operably linked.

17. A vector, wherein the vector comprises the enhanced promoter sequence according to claim 11.

18. The vector according to claim 17, wherein the promoter is an EF1α promoter, the nucleotide sequence of which is as shown in SEQ ID NO: 3; or the promoter is a hCMV promoter, the nucleotide sequence of which is as shown in SEQ ID NO: 4; or the promoter is a β-actin promoter, the nucleotide sequence of which is as shown in SEQ ID NO: 5.

19. The vector according to claim 17, wherein the vector is a eukaryotic expression vector, selected from the group consisting of: a transient expression vector, a virus expression vector and a transposition vector.

20. The vector according to claim 17, wherein the vector is a vector for integrating an expression cassette of a gene of interest into the genome of a host cell, or is a transposition vector comprising a transposable element selected from the group consisting of: piggyback, sleeping beauty, frog prince, Tn5 and Ty.

21. The vector according to claim 19, wherein the transposition vector sequentially contains the enhanced promoter sequence, one or more restriction sites or optional coding sequence(s) of interest, and a transcription termination sequence between the 5′ LTR and the 3′ LTR, that are operably linked.

22. The vector according to claim 21, wherein
a coding sequence of a transposase and its promoter sequence are operably linked at the 3′ end of the 3′ LTR;
the nucleic acid sequence of the vector is as shown in SEQ ID NO: 12;
the transcription termination sequence is SV40 polyA transcription termination sequence.

23. The vector according to claim 19, wherein,
the gene of interest is the coding sequence of a PD-1 single-chain antibody; and/or
the restriction site is selected from the group consisting of: an AscI restriction site, an XbaI restriction site, a PvuI restriction site, an EcoRI restriction site and a SalI restriction site.

24. A T cell, wherein the T cell comprises the enhanced promoter sequence according to claim 11, a nucleic acid construct comprising the enhanced promoter sequence, or a vector comprising the enhanced promoter sequence or the nucleic acid construct.

25. The T cell according to claim 24, wherein the genome of the T cell is integrated with an expression cassette, and the enhanced promoter sequence according to claim 11 is used in the expression cassette as a promoter to drive the expression of an exogenous gene of interest.

26. The T cell according to claim 24, wherein the promoter is an EF1α promoter, the nucleotide sequence of which is as shown in SEQ ID NO: 3; or the promoter is a hCMV promoter, the nucleotide sequence of which is as shown in SEQ ID NO: 4; or the promoter is a β-actin promoter, the nucleotide sequence of which is as shown in SEQ ID NO: 5.

27. A T cell stably expressing an antibody, wherein the genome of the T cell is integrated with an expression cassette comprising the enhanced promoter sequence and a coding sequence of the antibody operably linked to the promoter sequence.

28. The T cell according to claim 27, wherein the antibody is selected from the group consisting of: a PD-1 antibody, a PD-L1 antibody, a CTLA4 antibody, a CD40 antibody, a CD40L antibody, a CD47 antibody, a CD137 antibody, a CD28 antibody, a CD27 antibody, a OX40 antibody, a DNAM-1 antibody, a GITR antibody, an ICSOS antibody, a 2B4 antibody, a CD160 antibody, a TIM3 antibody, a LAG3 antibody, a BTLA antibody and a TIGIT antibody.

29. The T cell according to claim 27, wherein the antibody is a PD-1 antibody, and the amino acid sequence of the PD-1 antibody is as shown in SEQ ID NO: 10, or the coding sequence thereof is as shown in SEQ ID NO: 11.

30. The T cell according to claim 27, wherein the promoter is an EF1α promoter, the nucleotide sequence of which is as shown in SEQ ID NO: 3; or the promoter is a hCMV promoter, the nucleotide sequence of which is as shown in SEQ ID NO: 4; or the promoter is a β-actin promoter, the nucleotide sequence of which is as shown in SEQ ID NO: 5.


SEQUENCE LISTING (12 sequences)

SEQ ID NO: 1 - DNA, 144 bp, Artificial Sequence
  Description of Artificial Sequence Nucleotide sequence of sDTS
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaacca

SEQ ID NO: 2 - DNA, 211 bp, Artificial Sequence
  Description of Artificial Sequence Nucleotide sequence of sDTS-LTR
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccattaaccctagaaagataatcatattgtgacgtacgttaaagataatcatgcgtaaaattgacgcatg

SEQ ID NO: 3 - DNA, 545 bp, Artificial Sequence
  Description of Artificial Sequence Nucleotide sequence of EF1alpha promoter
aggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 4 - DNA, 584 bp, Artificial Sequence
  Description of Artificial Sequence Nucleotide sequence of hCMV promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 5 - DNA, 378 bp, Artificial Sequence
  Description of Artificial Sequence Nucleotide sequence of beta-Actin promoter
tcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtcc

SEQ ID NO: 6 - DNA, 600 bp, Artificial Sequence
  Description of Artificial Sequence Nucleotide sequence of nanoluciferase
atgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 7 - protein, 199 aa, Artificial Sequence
  Description of Artificial Sequence Amino acid sequence of nanoluciferase
MNSFSTSAFGPVAFSLGLLLVLPAAFPAPVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 8 - DNA, 720 bp, Artificial Sequence
  Description of Artificial Sequence Nucleotide sequence of EGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 9 - protein, 239 aa, Artificial Sequence
  Description of Artificial Sequence Amino acid sequence of EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 10 - DNA, 1488 bp, Artificial Sequence
  Description of Artificial Sequence Nucleotide sequence of anti-PD-1 antibody
atggaagccccagctcagcttctcttcctcctgctactctggctcccagataccaccggagaaattgtgttgacacagtctccagccaccctgtctttgtctccaggggaaagagccaccctctcctgcagggccagcaaaggtgtcagtacatctggctatagttatttgcactggtatcaacagaaacctggccaggctcccaggctcctcatctatcttgcatcctacctagaatctggcgtcccagccaggttcagtggtagtgggtctgggacagacttcactctcaccatcagcagcctagagcctgaagattttgcagtttattactgtcagcacagcagggaccttccgctcacgttcggcggagggaccaaagtggagatcaaaggtggaggcggttcaggcggaggtggcagcggcggtggcgggtcgcaggtgcagctggtgcagtccggcgtggaggtgaagaagcctggcgcctccgtcaaggtgtcctgtaaggcctccggctacaccttcaccaactactacatgtactgggtgcggcaggccccaggccagggactggagtggatgggcggcatcaacccttccaacggcggcaccaacttcaacgagaagttcaagaaccgggtgaccctgaccaccgactcctccaccacaaccgcctacatggaactgaagtccctgcagttcgacgacaccgccgtgtactactgcgccaggcgggactaccggttcgacatgggcttcgactactggggccagggcaccaccgtgaccgtgtcctccgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 11 - protein, 495 aa, Artificial Sequence
  Description of Artificial Sequence Amino acid sequence of anti-PD-1 antibody
MEAPAQLLFLLLLWLPDTTGEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - DNA, 5037 bp, Artificial Sequence
  Description of Artificial Sequence Nucleotide sequence of pNB338B-E vector
ggcgcgccttaaccctagaaagataatcatattgtgacgtacgttaaagataatcatgcgtaaaattgacgcatgtctagaggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgtcgaccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcatgcgtcaattttacgcagactatctttctagggttaaatcgatttagaagcagctctggcacatgtcgatgttgtgctcgcggcagatcaccttcttgcacttcttgcagctggcgctggccttgcggcggatcttgctggggcagtaggtgcagtaggtgcgcttcttcatcacgggctcctcggtgctgtcgtcgctggtgccgggcacctccttgggcaggatgttgctgatgttgtcgcgcaggtagcgcttcagggtgggggcctccaggcgcttgcgcatgaagctgctggtcaggcccatgtacaggttgcgcatgaacttcttgcggctctgcaccttctcgcccttgctgctcacgttgtggctgtagatgatgaagctgttgatgcaggcgatgttgatcatgccgtacagcagggccatgggccagcggttggtcttgcggctgcaggtcatcacgctgcacatctggtccagggtgtccacgccgcccttggtctggttgtagtacatcaccatctggggcttgccggtgctctcgttgatgctggcgtcctcgtcgcagctgctcagcaggtacaccatcttggcgggcttgggcttgtagctcaccagggtcagggggccgtcgaagcagaacatgctggtgcccacggggcggctgcggctgttcttcagcacctcggggatctcgcgcttgttgctgcgcacggtgcccacgatggtcagcttgtagggctcctgcagcaggttcttggccagggggatgctggtgaaccagttgtcgcaggtgatgttgcggcagctgccgtgcacgggcttgctcagctccttcacgtagtactcgcccaggggcacgccgttggtctgggtgccgcggcccaggtagggcatgccgttgatcatgtacttggtgccgctgtcgcacatcatcaggatcttgatgccgtacttgctgggcttgttggggatgtacacgcggaaggggcagcggccgcggaagcccagcagctgctcgtcgatggtcaggtgggcgccgggggtgtagttctggatgcactggtggatgaacaggtcccagatcttgcgcacgggggtgaacacgtcgttctcgcgcagggtggggcggatgctcttgtcgtccatgcgcaggcagcggatcaggaagtcgaagcggtcgcggctcatcacgctcacgtacaccatgctcaggctgcggtcgaacaggtcgtcggtgctcatgtggttgtccttgcgcacggcggtcatcaccaggatgccgaagaaggcgtagatctcgtcctcgttggtgtcgcggaaggtggcgctggtcatgctctcgcggcgcttcaggctgatctcggcgttggtccacttcacgatctcgctgatgatctcgtcggtgaagaacagcttgaagcacagcagggggtcgtagatgttgcggcacatgcgggtggggccgcgctggctgcgcacgatgttcagggcgctcacgcggctgcggcgggtgggcttgctggtgctccagcagtgcttgttcttgccgcggatggtgcgctggggcagggtcaggatgcggttgctggccaggctgctgccgggctgctcgatcacgttctgctcgtccaggatctcgctgccgctgctggtgggctgcacctcgtgcacctcgtcgatgaaggcctcctcggtgtcgctctgcacgtcgtcctcgctcacgtggtcgctcacctcgctgtcgctgtcctcgcccaccagctcgtcgtcgctctgcagcagggcgctcaggatgtgctcgtcgtccaggctgctgccgtccaacttgaccctcttggcagcagggcccatggtggcaagcttgatctgcgttctacggtggtcagaccgaagactgcgacggtaccgacgctggtcgcgcctcttatacccacgtagaacgcagctcagccaatagaatgagtgccaatatggaatttccaggggaaaaccggggcggtgttacgttttggctgccctttcacttcccattgacgtgtattggctcgagaacggtactttcccattaatcagctatgggaaagtaccgtttaaaggtcacgttgcattagtttcaatagcccattgacgtcaatggtgggaaagtacatggcgttttaatttaatggctggaaaaacccaatgactcacccctattgaccttatgtacgtgccaataatgggaaaaacccattgactcaccccctattgaccttttgtactgggcaaaacccaatggaaagtccctattgactcagtgtacttggctccaatgggactttcctgttgattcacccctattgaccttatgtactgggcaaaacccattggaaagtccctaatgactcagtatatttaattaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctctctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagtacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggttcgtctcgtcccggtctcctcccatgcatg